(19) United States
(12) Patent Application Publication
(10) Pub. No.: US 2024/0090878 A1
(43) Pub. Date: Mar. 21, 2024

(54) AEROSOL SAMPLE COLLECTION DEVICE

(71) Applicant: Proveris Scientific Corporation, Hudson, MA (US)

(72) Inventor: Dino John FARINA, Sudbury, MA (US)

(21) Appl. No.: 18/483,933

(22) Filed: Oct. 10, 2023

Related U.S. Application Data
(63) Continuation of application No. PCT/US2022/024268, filed on Apr. 11, 2022.
(60) Provisional application No. 63/173,791, filed on Apr. 12, 2021.

(51) Int. Cl. A61B 10/00

(52) U.S. Cl. CPC A61B 10/00; A61B 2010/0087

(57) ABSTRACT

This disclosure relates generally to devices to collect aerosol sample and methods thereof.

### CROSS-REFERENCE

This application is a continuation of International Patent Application No. PCT/US2022/024268, filed on Apr. 11, 2022, which claims the benefit of U.S. Provisional Patent Application No. 63/173,791, filed on Apr. 12, 2021, each of which is incorporated herein by reference in its entirety.

### BACKGROUND

Numerous respiratory viruses, such as coronaviruses (e.g., severe acute respiratory syndrome coronavirus-2 (SARS-CoV-2)), measles virus, varicella-zoster virus, adenovirus, influenza (e.g., H1N1), rhinovirus, as well as respiratory bacteria, such as Mycobacterium tuberculosis and Bordetella pertussis, are transmitted from an infected subject in particle droplets. These virus or bacteria particle droplets are generated when an infected person for example, coughs or sneezes. Collecting these particle droplets can facilitate diagnosis of infection. Current sample collection methods for respiratory viruses and bacteria require nasal sampling such as nasopharyngeal swab which can be invasive and uncomfortable. Moreover, nasal sampling may have poor signal-to-noise ratio, depending on the swabbing skills of the subject. Poor signal-to-noise ratio often leads to false diagnosis results.

### SUMMARY

There is a great unmet need for new sampling devices that are non-invasive, easy for a subject to use, and have a high signal-to-noise ratio that can generate more accurate diagnosis results. The present disclosure meets this unmet need by providing devices for collecting aerosol samples from a subject which address these challenges.

In some aspects, a device for collecting an aerosol sample from a subject is disclosed, comprising (a) a conduit defining an inner space for the passage of exhaled air, wherein the conduit comprises (i) a first opening for receiving exhaled air from the subject, (ii) a second opening for expelling at least a portion of the exhaled air, and (iii) a long axis; and (b) a filter for collecting the aerosol sample from the exhaled air, wherein the filter is positioned between the first opening and the second opening and is substantially perpendicular to the long axis, and wherein the filter is an electrostatically charged filter. In some embodiments, the filter exhibits a low airflow resistance.

In some aspects, a device for collecting an aerosol sample from a subject is disclosed, comprising (a) a conduit defining an inner space for the passage of exhaled air, wherein the conduit comprises (i) a first opening for receiving the exhaled air from the subject, (ii) a second opening for expelling at least a portion of the exhaled air, and (iii) a long axis; and (b) a filter for collecting the aerosol sample from the exhaled air, wherein the filter is positioned between the first opening and the second opening and is substantially perpendicular to the long axis, and wherein the filter exhibits a low airflow resistance.

In some embodiments, the filter exhibits an airflow resistance of less than about 1.5 cm H2O/L/sec at flow rates of about 14 L/sec. In some embodiments, the device further comprises a filter support, wherein the filter support is positioned between the first opening and the second opening and is substantially perpendicular to the long axis, and wherein the filter is secured to the filter support. In some embodiments, the filter support comprises a filter web. In some embodiments, the filter web comprises one or more opening. In some embodiments, the filter web comprises resin. In some embodiments, the filter web comprises polypropylene or polyethylene terephthalate. In some embodiments, the conduit comprises resin. In some embodiments, the conduit comprises polypropylene or polyethylene terephthalate. In some embodiments, the filter support further comprises a shoulder. In some embodiments, the filter is secured between the shoulder and the filter web. In some embodiments, the filter support is permanently secured to the conduit. In some embodiments, the filter support is sonically welded to the conduit. In some embodiments, a cross-sectional area of the inner space between the first opening and the second opening is constant. In some embodiments, the length of the conduit between the first opening and the second opening is about 100 to 150 millimeters. In some embodiments, the diameter of the conduit is about 10 to 20 millimeters. In some embodiments, the diameter of the conduit is about 16 millimeters.

In some embodiments, the device further comprises a first removable cap, wherein the first removable cap is configured to secure the first opening. In some embodiments, the device further comprises a second removable cap, wherein the second removable cap is configured to secure the second opening. In some embodiments, the first opening has a threaded surrounding wall that mates with a threaded surrounding wall of the first removable cap. In some embodiments, the second opening has a threaded surrounding wall that mates with a threaded surrounding wall of the second removable cap. In some embodiments, the first removable cap and the second removable cap comprise different colors. In some embodiments, the device further comprises a removable film, wherein the removable film covers at least a portion of an outside wall of the conduit.

In some embodiments, the filter exhibits an airflow resistance of less than about 1.0, less than about 0.9, less than about 0.8, less than about 0.7, less than about 0.6, less than about 0.5, less than about 0.4, less than about 0.3, less than about 0.2, or less than about 0.1 cm H2O/L/sec at flow rates of about 14 L/sec. In some embodiments, the filter comprises a non-woven synthetic fiber or a nanofiber. In some embodiments, the filter comprises a material that is non-chemically reactive with the aerosol sample. In some embodiments, the material is polypropylene, polyester, or nylon. In some embodiments, the filter is hydrophobic. In some embodiments, the filter exhibits at least about 95%, at least about 96%, at least about 97%, at least about 98%, at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency. In some embodiments, the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency. In some embodiments, the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency. In some embodiments, the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% viral filtration efficiency.

In some embodiments, the filter is positioned near the second opening. In some embodiments, the filter is positioned such that the filter can be immersed in a medium. In some embodiments, the medium has a volume of less than about 20 milliliters. In some embodiments, the medium has a volume of less than 5 milliliters. In some embodiments, the medium has a volume of about 3 milliliters.

In some embodiments, the filter comprises a sensor configured to detect a molecule or a pathogen. In some embodiments, the sensor detects the molecule or the pathogen by color, an optical signal, an electronic signal, a chemical signal, or by artificial intelligence. In some embodiments, the sensor detects the molecule or the pathogen by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy. In some embodiments, the sensor is configured to detects a bacterium, a virus or a viral particle. In some embodiments, the virus or viral particle is SARS-CoV-2, a variant or a mutant thereof.

In some embodiments, the device further comprises a detector to detect strength of breath and/or loudness of voice by the subject. In some embodiments, the detector is positioned between the first opening and the second opening. In some embodiments, the detector comprises a perforated membrane. In some embodiments, the detector is attached to an outside wall of the conduit. In some embodiments, the detector is a detecting system that comprises (a) a sensor for collecting information regarding the strength of the breath and/or the loudness of the voice by the subject, and providing the information to a processor; (b) the processor for processing the information provided by the sensor; and (c) a structure configured to allow the sensor to attach to the conduit. In some embodiments, the device is configured to capture speech-generated droplets provided by a subject while speaking. In some embodiments, the device further comprises a blocking structure to prevent backflow, wherein the blocking structure is positioned between the filter and the second opening. In some embodiments, the blocking structure is a second filter or a one-way valve. In some embodiments, the device further comprises a mouthpiece, wherein the mouthpiece is attached to the first opening to receive the exhaled air.

In some aspects, a device for collecting an aerosol sample from a subject is disclosed, comprising (a) a conduit defining an inner space for the passage of exhaled air, wherein the conduit has a first opening for receiving the exhaled air from the subject; (b) a filter positioned in the inner space; and (c) a detector to detect strength of breath, loudness of voice, or both, wherein the detector is positioned in the inner space. In some embodiments, the detector is positioned between the first opening and the filter.

In some aspects, a device configured to collect speech-generated droplets from a subject is disclosed, comprising: (a) a conduit defining an inner space for the passage of exhaled air comprising the speech-generated droplets, wherein the conduit has an opening for receiving the exhaled air from the subject; and (b) a filter for collecting the speech-generated droplets from the exhaled air, wherein the filter is positioned in the inner space; wherein the speech-generated droplets are provided from speaking by the subject.

In some embodiments, the exhaled air is provided by speaking by the subject. In some embodiments, the device further comprises a detector to detect strength of breath, loudness of voice, or both. In some embodiments, the detector is positioned in the inner space. In some embodiments, the detector comprises a perforated membrane. In some embodiments, the detector is attached to an outside wall of the conduit. In some embodiments, the detector is a detecting system that comprises (a) a sensor for collecting information regarding the strength of the breath and/or the loudness of the voice by the subject, and providing the information to a processor; (b) the processor for processing the information provided by the sensor; and (c) a structure configured to allow the sensor to attach to the conduit of the devices. In some embodiments, the filter exhibits a low airflow resistance. In some embodiments, the filter exhibits an airflow resistance of less than about 1.5 cm H2O/L/sec at flow rates of about 14 L/sec. In some embodiments, the filter is electrostatically charged. In some embodiments, the device further comprises a long axis and a second opening for expelling at least a portion of the exhaled air, wherein the filter is positioned between the first opening and the second opening. In some embodiments, the device further comprises a filter support, wherein the filter support is positioned between the first opening and the second opening and is substantially perpendicular to the long axis, and wherein the filter is secured to the filter support. In some embodiments, the filter support comprises a filter web. In some embodiments, the filter web comprises one or more opening. In some embodiments, the filter web comprises resin. In some embodiments, the filter web comprises polypropylene or polyethylene terephthalate. In some embodiments, the conduit comprises resin. In some embodiments, the conduit comprises polypropylene or polyethylene terephthalate. In some embodiments, the filter support further comprises a shoulder. In some embodiments, the filter is secured between the shoulder and the filter web. In some embodiments, the filter support is permanently secured to the conduit. In some embodiments, the filter support is sonically welded to the conduit. In some embodiments, a cross-sectional area of the inner space between the first opening and the second opening is constant. In some embodiments, the length of the conduit between the first opening and the second opening is about 100 to 150 millimeters. In some embodiments, the diameter of the conduit is about 10 to 20 millimeters. In some embodiments, the device further comprises a first removable cap, wherein the first removable cap can secure the first opening. In some embodiments, the device further comprises a second removable cap, wherein the second removable cap can secure the second opening. In some embodiments, the first opening has a threaded surrounding wall that mates with a threaded surrounding wall of the first removable cap. In some embodiments, the second opening has a threaded surrounding wall that mates with a threaded surrounding wall of the second removable cap. In some embodiments, the first removable cap and the second removable cap comprise different colors. In some embodiments, the device further comprises a removable film, wherein the removable film covers at least a portion of an outside wall of the conduit.

In some embodiments, the filter exhibits an airflow resistance of less than about 1.0, less than about 0.9, less than about 0.8, less than about 0.7, less than about 0.6, less than about 0.5, less than about 0.4, less than about 0.3, less than about 0.2, or less than about 0.1 cm H2O/L/sec at flow rates of about 14 L/sec. In some embodiments, the filter comprises a non-woven synthetic fiber or a nanofiber. In some embodiments, the filter comprises a material that is non-chemically reactive with the aerosol sample. In some embodiments, the material is polypropylene, polyester, or nylon. In some embodiments, the filter is hydrophobic. In some embodiments, the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% bacterial filtration efficiency. In some embodiments, the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency. In some embodiments, the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency. In some embodiments, the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% viral filtration efficiency. In some embodiments, the filter is positioned near the second opening. In some embodiments, the filter comprises a sensor that detects a molecule or pathogen. In some embodiments, the sensor detects the molecule or pathogen by a color signal, an optical signal, an electronic signal, a chemical signal, or by artificial intelligence. In some embodiments, the sensor detects the molecule or pathogen by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy. In some embodiments, the sensor detects a bacterium, a virus or a viral particle. In some embodiments, the device further comprises a blocking structure to prevent backflow, wherein the blocking structure is positioned between the filter and the second opening. In some embodiments, the blocking structure is a second filter or a one-way valve. In some embodiments, the device further comprises a mouthpiece, wherein the mouthpiece is attached to the first opening to receive the exhaled air.

In some aspects, a method for collecting an aerosol sample from a subject is disclosed, comprising obtaining or having obtained any one of the devices disclosed herein; and receiving exhaled air from the subject into the inner space through the first opening.

In some embodiments, the exhaled air is provided by speaking by the subject. In some embodiments, the speaking comprises repeatedly speaking a phrase with an average loudness of at about 59 dB_{B} at a distance of about 30 centimeters. In some embodiments, the speech of the subject comprises repeatedly speaking a phrase with a loudness of about 85 dB_{B} or less at a distance of 30 centimeters. In some embodiments, the aerosol sample comprises speech-generated droplets. In some embodiments, the speech-generated droplets have a size from about 1 μm to about 150 μm. In some embodiments, the speech-generated droplets have a size from about 10 μm to about 100 μm. In some embodiments, the method further comprises measuring loudness of the speaking. In some embodiments, the method further comprises securing a second cap to a second opening. In some embodiments, the method further comprises adding a medium to the inner space of the device, whereby the filter is immersed in the medium. In some embodiments, the method further comprises securing a first cap to a first opening. In some embodiments, the method further comprises detecting a molecule or pathogen from the filter. In some embodiments, the molecule or pathogen is detected by color, an optical signal, an electronic signal, a chemical signal, or by artificial intelligence. In some embodiments, the molecule or pathogen is detected by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy.

In some aspects, a method for collecting an aerosol sample from a subject is disclosed, comprising providing a device comprising a filter for collecting the aerosol sample from the subject, and receiving exhaled air from the subject, wherein the exhaled air is provided by speaking by the subject.

In some embodiments, the method further comprises detecting loudness of the speaking. In some embodiments, the speaking comprises repeatedly speaking a phrase with an average loudness of about 59 dB_{B} at a distance of about 30 centimeters. In some embodiments, the speech of the subject comprises repeatedly speaking a phrase with a loudness of about 85 dB_{B} or less at a distance of about 30 centimeters. In some embodiments, the aerosol sample comprises speech-generated droplets. In some embodiments, the speech-generated droplets have a size from about 1 μm to about 150 μm. In some embodiments, the speech-generated droplets have a size from about 10 μm to about 100 μm. In some embodiments, the filter is electrostatically charged. In some embodiments, the filter exhibits a low airflow resistance. In some embodiments, the method further comprises adding a medium to the device such that the filter is immersed in the medium. In some embodiments, the method further comprises detecting a molecule or pathogen from the filter. In some embodiments, the molecule or pathogen is detected by a color signal, an optical signal, an electronic signal, a chemical signal, or by artificial intelligence. In some embodiments, the molecule or pathogen is detected by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy. In some embodiments, the molecule or pathogen is a bacterium, a virus or a viral particle. In some embodiments, the virus or viral particle is SARS-CoV-2, a variant or a mutant thereof.

In some aspects, a detecting system for detecting strength of breath and/or loudness of voice of a subject is disclosed, comprising (a) a sensor for collecting information regarding the strength of the breath and/or the loudness of the voice of the subject, and providing the information to a processor; (b) the processor for processing the information provided by the sensor; and (c) a structure configured to allow the sensor to attach to the conduit of any one of the devices disclosed herein. In some embodiments, the sensor comprises a sound amplifier. In some embodiments, the structure is in an arch-shape that fits the conduit of any one of the devices disclosed herein. In some embodiments, the processor comprises at least one indicator for indicating the strength of the breath and/or the loudness of the voice of the subject based on the information provided by the sensor.

In some aspects, a system for collecting an aerosol sample from a subject is disclosed, comprising (a) a conduit defining an inner space for the passage of exhaled air, wherein the conduit has a first opening for receiving the exhaled air from the subject; (b) a filter positioned in the inner space; and (c) any of the detecting system disclosed herein.

In some aspects, a kit for collecting an aerosol sample from a subject is disclosed, comprising any one of the devices disclosed herein, and instructions for collecting the aerosol sample.

In some embodiments, the kit further comprises a resealable bag. In some embodiments, the kit further comprises a medium. In some embodiments, the medium has a volume of at least 3 milliliters. In some embodiments, the medium comprises a proteinaceous solution. In some embodiments, the proteinaceous solution comprises beef extract. In some embodiments, the beef extract has a concentration between 1.5% and 3.0%. In some embodiments, the beef extract comprises veal infusion broth or fetal bovine serum. In some embodiments, the medium further comprises an antibiotic. In some embodiments, the antibiotic is gentamicin sulfate. In some embodiments, the medium further comprises an antifungal. In some embodiments, the antifungal is amphotericin B. In some embodiments, the medium further comprises a salt-based solution. In some embodiments, the salt-based solution comprises Hank's Balanced Salt Solution (HBSS). In some embodiments, the salt-based solution further comprises calcium and magnesium ions. In some embodiments, the medium further comprises an eluting solution. In some embodiments, the eluting solution comprises glycine. In some embodiments, the eluting solution further comprises at least one amphoteric amino acid. In some embodiments, the eluting solution further comprises a sodium polyphosphate solution. In some embodiments, the kit further comprises a unique barcode label for traceability. In some embodiments, the unique barcode label is attached to the device. In some embodiments, the kit further comprises any of the detecting system disclosed herein.

### Incorporation By Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a non-limiting example of an aerosol sample collection device, according to embodiments of the disclosure.

FIG. 2 illustrates a non-limiting example of a filter of an aerosol sample collection device, according to embodiments of the disclosure.

FIG. 3 illustrates another non-limiting example of an aerosol sample collection device, according to embodiments of the disclosure.

FIG. 4A illustrates a non-limiting example of the expanded view of the system for collecting an aerosol sample from a subject with a mouthpiece.

FIG. 4B illustrates a non-limiting example of an assembly view of the system of FIG. 4A.

FIG. 5A illustrates a non-limiting example of an expanded view of the detecting system for detecting the strength of breath and/or the loudness of voice.

FIG. 5B illustrates a non-limiting example of an assembly view of the detecting system of FIG. 5A.

FIG. 6A illustrates a non-limiting example of an expanded view of the system for collecting an aerosol sample from a subject, comprising the detecting system and the conduit.

FIG. 6B illustrates a non-limiting example of an assembly view of the system of FIG. 6A that is ready for use, that is, for a subject to breathing into or speaking into.

FIG. 7A illustrates a non-limiting example of the device after use, with the optional mouthpiece and the optional detecting system removed.

FIG. 7B illustrates a non-limiting example of the device with one cap installed on the second opening of the conduit, that is, the opening that is near the filter. The device is ready for solvent addition.

FIG. 7C illustrates a non-limiting example of the device that is ready for storage, with two caps installed on both openings.

### DETAILED DESCRIPTION

This disclosure relates generally to devices for collecting aerosol samples from a subject and methods to collect aerosol samples from a subject.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The terms “about” or “approximately” mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, the term “aerosol sample” refers to a gaseous substance comprising diminutive or lightweight body of matters. The sample may comprise, without limitation, dust motes, exfoliated skin cells, fibers, spores, vegetative cells, mists, condensates, virus particles, bacteria, yeast, mucous droplets, microdroplets of saliva and bronchial secretions, pollen grains, fly ash, smog condensate, smoke, fumes, dirt, fogs (as in industrial or agricultural spray application), salt, silicates, metallic particulate toxins, tar, combustion derived nanoparticles, particulate toxins, and the like. The sample may comprise a composition of both solid and liquid matter.

As used herein, the term “speech-generated droplets” refers to oral fluid droplets produced by speaking. The oral fluid droplets may comprise an aerosol sample. The speech-generated droplets may have a size from about 1 μm to about 500 μm, from about 1 μm to about 400 μm, from about 1 μm to about 300 μm, from about 1 μm to about 200 μm, from about 1 μm to about 100 μm, from about 10 μm to about 300 μm, from about 10 μm to about 200 μm, from about 10 μm to about 100 μm, from about 10 μm to about 90 μm, from about 10 μm to about 80 μm, from about 10 μm to about 70 μm, from about 10 μm to about 60 μm, from about 10 μm to about 50 μm, from about 10 μm to about 40 μm, from about 10 μm to about 30 μm, from about 10 μm to about 20 μm, or from about 1 μm to about 30 μm. Compared to droplets generated by normal breathing, speech-generated droplets are emitted at relatively high rate, such as from about 1,000 to about 10,000 droplets per second.

As used herein, the term “substantially perpendicular” refers to sufficiently perpendicularly such that performance of a component is the same as though the component is positioned precisely perpendicular.

As used herein, the term “nonwoven” refers to a type of fabric that is bonded together rather than being spun and woven into a cloth.

The terms “airflow resistance” and “resistance to airflow” are used interchangeably herein, and refer to the resistance to airflow through a filter (e.g., a droplet capturing filter described herein).

The terms “low airflow resistance” as used herein refers to an airflow resistance close to zero. The airflow resistance is measured according to the National Institute for Occupational Health & Safety (NIOSH) Standard Respirator Testing Procedures TEB-APR-STP-0003.

The term “efficiency” as used herein refers to the level of filtration protection or function that a filter can deliver. The efficiency of the filter is normally expressed as a reflection of the number of microorganisms that pass through the filter media when it is challenged. This filter is then described as being X % efficient. The X % is an expression of the number of organisms penetrating the filter when challenged by an aerosol containing 1,000,000 microorganisms.

The section headings used herein are for organizational purposes only and are not to be construed as limiting the subject matter described.

### Aerosol Sample Collection Devices

Numerous respiratory viruses such as coronaviruses (e.g., severe acute respiratory syndrome coronavirus-2 (SARS-CoV-2), measles virus, varicella-zoster virus, and adenovirus, influenza (e.g., H1N1), and rhinovirus, as well as respiratory bacteria such as Mycobacterium tuberculosis and Bordetella pertussis are transmitted from an infected subject in particle droplets. These virus or bacteria particle droplets are generated when an infected person for example, coughs or sneezes. Collecting these particle droplets can facilitate diagnosis of infection. Current sample collection methods for respiratory viruses and bacteria require nasal sampling such as nasopharyngeal swab which can be invasive and uncomfortable. Moreover, nasal sampling may have poor signal-to-noise ratio, depending on the swabbing skills of the subject. Poor signal-to-noise ratio often leads to false diagnosis results. Therefore, a great need exists for new sampling devices that are non-invasive, easy for a subject to use, and have a high signal-to-noise ratio that can generate more accurate diagnosis results.

The present disclosure describes devices for collecting aerosol sample from a subject. Specifically, the present disclosure describes non-invasive devices for collecting the particle droplets from a subject by breathing into or speaking into the devices. The aerosol sample collected by the devices disclosed herein has a high signal-to-noise ratio and thus can facilitate more accurate diagnosis. In addition, the devices disclosed herein are not dependent on the swabbing skills of the subject. Thus, the devices disclosed herein are easy and comfortable for a subject to use.

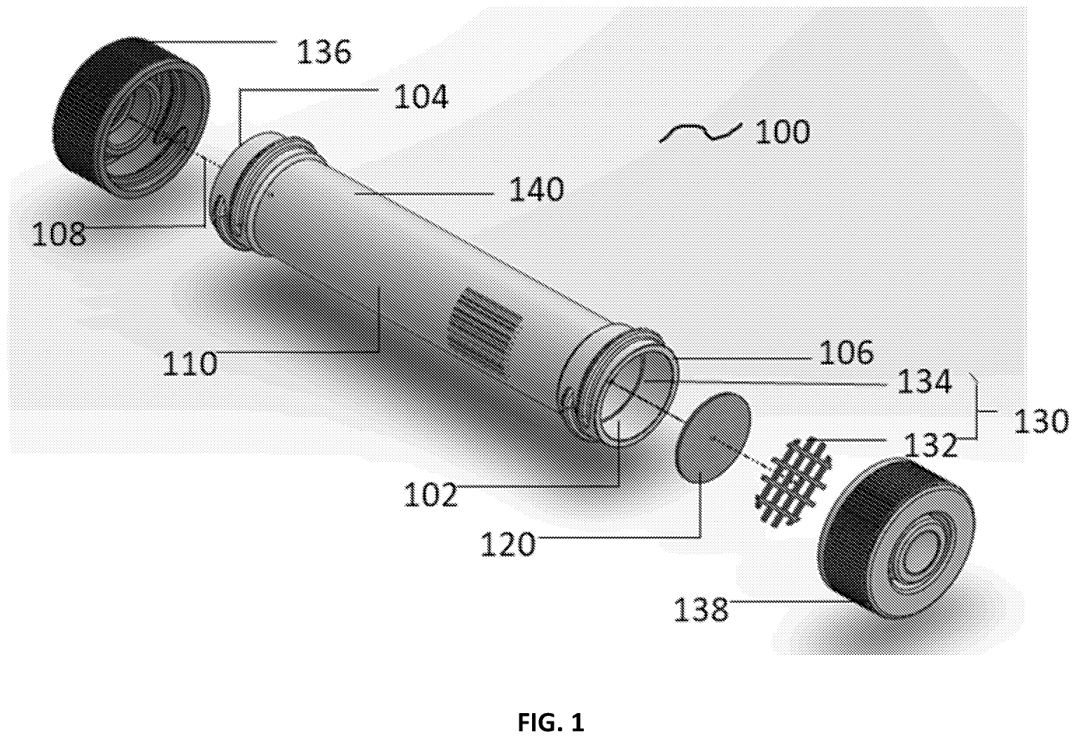

As shown in FIG. 1, the present disclosure describes a device 100 for collecting an aerosol sample from a subject, comprising a conduit 110 defining an inner space 102 for the passage of exhaled air, wherein the conduit 110 comprises a first opening 104 for receiving the exhaled air from the subject, a second opening 106 for expelling at least a portion of the exhaled air, and a long axis 108; and a filter 120 for collecting an aerosol sample from the exhaled air, wherein the filter 120 is positioned between the first opening 104 and the second opening 106 and is substantially perpendicular to the long axis 108, wherein the filter 120 is an electrostatically-charged filter.

In some embodiments, a device 100 for collecting aerosol sample from a subject is disclosed herein, comprising a conduit 110 defining an inner space 102 for the passage of exhaled air, wherein the conduit 110 comprises a first opening 104 for receiving the exhaled air from the subject, a second opening 106 for expelling at least a portion of the exhaled air, and a long axis 108; and a filter 120 for collecting the aerosol sample from the exhaled air, wherein the filter 120 is positioned between the first opening 104 and the second opening 106 and is substantially perpendicular to the long axis 108, and wherein the filter 120 exhibits a low airflow resistance.

In some embodiments, the device 100 further comprises a filter support 130, wherein the filter support 130 is positioned between the first opening 104 and the second opening 106 and is substantially perpendicular to the long axis 108, and wherein the filter 120 is secured to the filter support 130. The filter support 130 may be any structure that can be used to position and fixate a filter. In some embodiments, the filter support 130 comprises a filter web 132. In some embodiments, the filter web 132 comprises one or more opening to allow substance to pass through, such as gas. The opening can be in any shape or size. In some embodiments, the filter web 132 comprises resin. In some embodiments, the filter web 132 comprises polypropylene or polyethylene terephthalate. In some embodiments, the conduit 110 comprises resin. In some embodiments, the conduit 110 comprises polypropylene or polyethylene terephthalate. In some embodiments, the filter web 132 is made from the same material as the conduit 110. In some embodiments, the filter web 132 is made from the same resin as the conduit 110. In some embodiments, the filter web 132 is made from the same polypropylene or polyethylene terephthalate as the conduit 110. In some embodiments, the filter support 130 further comprises a shoulder 134. The shoulder 134 may extend from the inner side wall of the conduit 110. The shoulder 134 may be used within the filter web 132 to position and fixate the filter 120. The shoulder 134 may be in the shape of a continuous ring. In some embodiments, the shoulder 134 may comprise multiple pieces, wherein the multiple pieces are configured in the inner space 102 of the conduit 110 such that the filter 120 can be place on the shoulder 134. In some embodiments, the multiple pieces are attached to different locations of the inner side wall of the conduit 110 but on the same cross section of the conduit 110. In some embodiments, the filter 120 is secured between the shoulder 134 and the filter web 132. In some embodiments, the filter support 130 is permanently secured to the conduit. In some embodiments, the filter support 130 is sonically welded to the conduit. In some embodiments, the cross-sectional area of the inner space 102 of the conduit between the first opening 104 and the second opening 106 is constant.

In some embodiments, the length of the conduit 100 between the first opening 104 and the second opening 106 is about 50 to about 300 millimeters. In some embodiments, the length of the conduit 100 between the first opening 104 and the second opening 106 is about 80 to about 250 millimeters. In some embodiments, the length of the conduit 110 between the first opening 104 and the second opening 106 is about 100 to about 200 millimeters. In some embodiments, the length of the conduit 100 between the first opening 104 and the second opening 106 is about 110 to about 190 millimeters. In some embodiments, the length of the conduit 100 between the first opening 104 and the second opening 106 is about 120 to about 180 millimeters. In some embodiments, the length of the conduit 100 between the first opening 104 and the second opening 106 is about 130 to about 170 millimeters. In some embodiments, the length of the conduit 100 between the first opening 104 and the second opening 106 is about 140 to about 160 millimeters. In some embodiments, the length of the conduit 100 between the first opening 104 and the second opening 106 is about 150 millimeters.

In some embodiments, the diameter of the conduit 110 is about 5 to about 30 millimeters. In some embodiments, the diameter of the conduit 110 is about 10 to about 20 millimeters. In some embodiments, the diameter of the conduit 110 is about 13 to about 17 millimeters. In some embodiments, the diameter of the conduit 110 is about 16 millimeters.

In some embodiments, the conduit 110 fits the requirements of an industry standard 10 mL centrifuge tube, including diameters and wall thickness requirements. In some embodiments, the conduit 110 may be volumetrically compatible with current CDC/FDA guidelines for COVID-19 sample collection/storage in the field by trained personnel. In some embodiments, the conduit 110 may be volumetrically compatible with current CDC/FDA guidelines for collecting/storing measles virus, varicella-zoster virus, Mycobacterium tuberculosis, adenovirus, influenza (e.g., H1N1), rhinovirus, and/or Bordetella pertussis. In some embodiments, the conduit 110 may be volumetrically compatible with current CDC/FDA guidelines for collecting/storing other aerosol samples. In some embodiments, the conduit 110 may have a 30 mL total volume. In some embodiments, the conduit 110 may have a 20 mL total volume. In some embodiments, the conduit 110 may have a 10 mL total volume.

In some embodiments, the device further comprises a first removable cap 136, wherein the first removable cap 136 can secure the first opening 104. In some embodiments, the device further comprises a second removable cap 138, wherein the second removable cap 138 can secure the second opening 106. In some embodiments, the first opening 104 has a threaded surrounding wall that mates with a threaded surrounding wall of the first removable cap 136. In some embodiments, the second opening 106 has a threaded surrounding wall that mates with a threaded surrounding wall of the second removable cap 138. In some embodiments, the first removable cap 136 has an indicator indicating that the cap is the first removable cap. In some embodiments, the second removable cap 138 has an indicator indicating that the cap is the second removable cap. In some embodiments, the indicator may be of color, symbol, or shapes. In some embodiments, the first removable cap 136 and the second removable cap 138 comprise different colors. In some embodiments, the first removable cap or the second removable cap fits the requirements of a cap of an industry standard 10 mL centrifuge tube, such as threads requirements. In some embodiments, the device 100 further comprises a removable film 140, wherein the removable film 140 covers at least a portion of an outside wall of the conduit 110. In some embodiments, the removable film 140 covers the whole outside wall of the conduit 110. The removable film 140 may be a tear-away film. The removable film 140 may be used to prevent against contamination from human contact (e.g., patient's lips and/or fingers) during sampling.

In some embodiments, the filter 120 exhibits a low airflow resistance. In some embodiments, the filter 120 exhibits an airflow resistance of less than about 1.5 cm H₂O/L/sec at flow rates of about 14 L/sec. In some embodiments, the filter 120 exhibits an airflow resistance of less than about 1.0, less than about 0.9, less than about 0.8, less than about 0.7, less than about 0.6, less than about 0.5, less than about 0.4, less than about 0.3, less than about 0.2, or less than about 0.1 cm H₂O/L/sec at flow rates of 14 L/sec. In some embodiments, the filter 120 comprises a non-woven synthetic fiber or a nanofiber. In some embodiments, the filter 120 comprises a material that is non-chemically reactive with the aerosol sample. In some embodiments, the material may be polypropylene, polyester, or nylon. In some embodiments, the filter 120 is hydrophobic. In some embodiments, the filter 120 exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% bacterial filtration efficiency. In some embodiments, the filter 120 exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency. In some embodiments, the filter 120 exhibits at least about 95%, at least about 96%, at least about 97%, at least about 98%, at least about 99%, at least about 99.1%, at least about 99.2%, at least about 99.3%, at least about 99.4%. at least about 99.5%, at least about 99.6%, at least about 99.7%, at least about 99.8%, or at least about 99.9% viral filtration efficiency. In some embodiments, the filter 120 exhibits at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency.

In some embodiments, the filter 120 is positioned near one of the openings 104, 106. In some embodiments, the filter 120 is positioned near the second opening 106. In some embodiments, the filter 120 is positioned such that the filter 120 can be immersed in a medium. The medium may have a volume of less than about 20 milliliters, a volume of less than about 10 milliliters, a volume of less than about 5 milliliters, or a volume of less than about 3 milliliters.

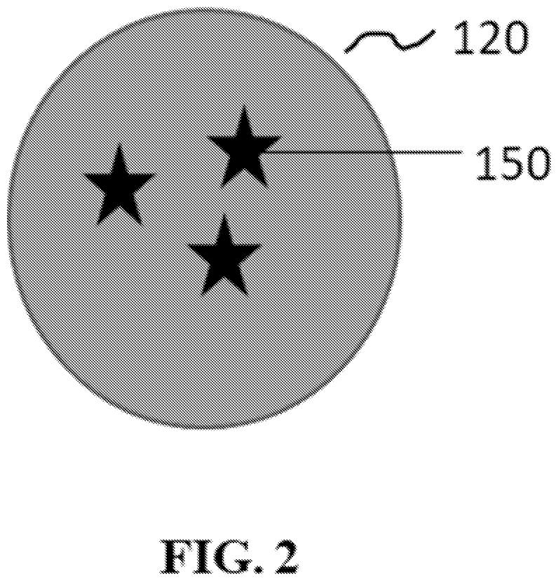

As shown in FIG. 2, in some embodiments, the filter 120 comprises a sensor 150 that detects a molecule or pathogen. In some embodiments, the sensor 150 of the device detects the molecule or pathogen by color, optical, electronic, chemical or artificial intelligence means. For example, the sensor 150 may detect the molecule or pathogen by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy. In some embodiments, the sensor 150 detects a bacterium, a virus, or a viral particle. In some embodiments, the virus or the viral particle is a coronavirus. In some embodiments, the coronavirus is SARS-COV-2, or a variant thereof, or a mutant thereof.

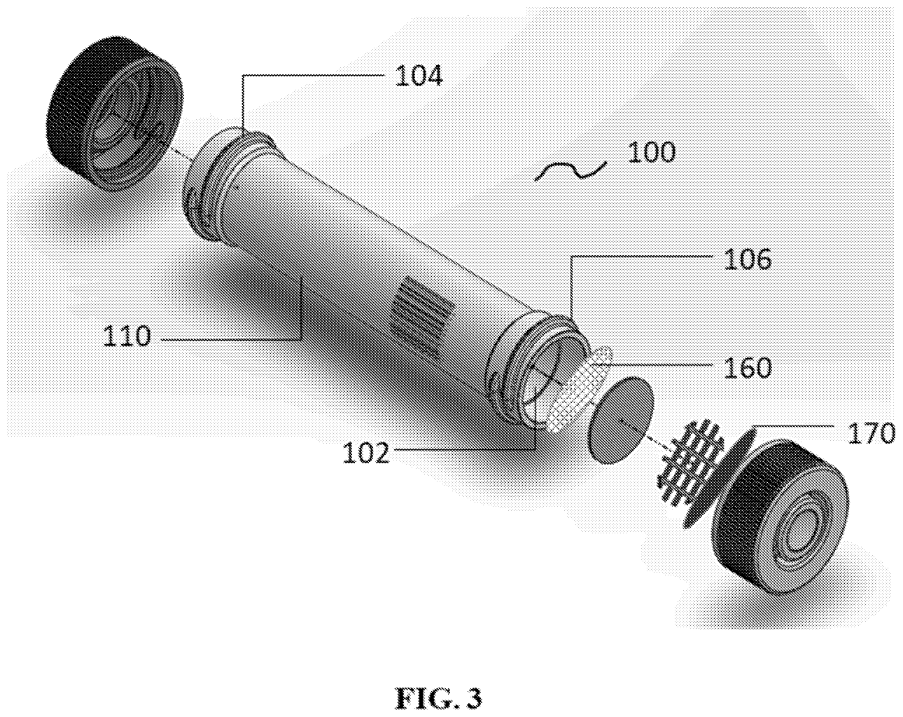

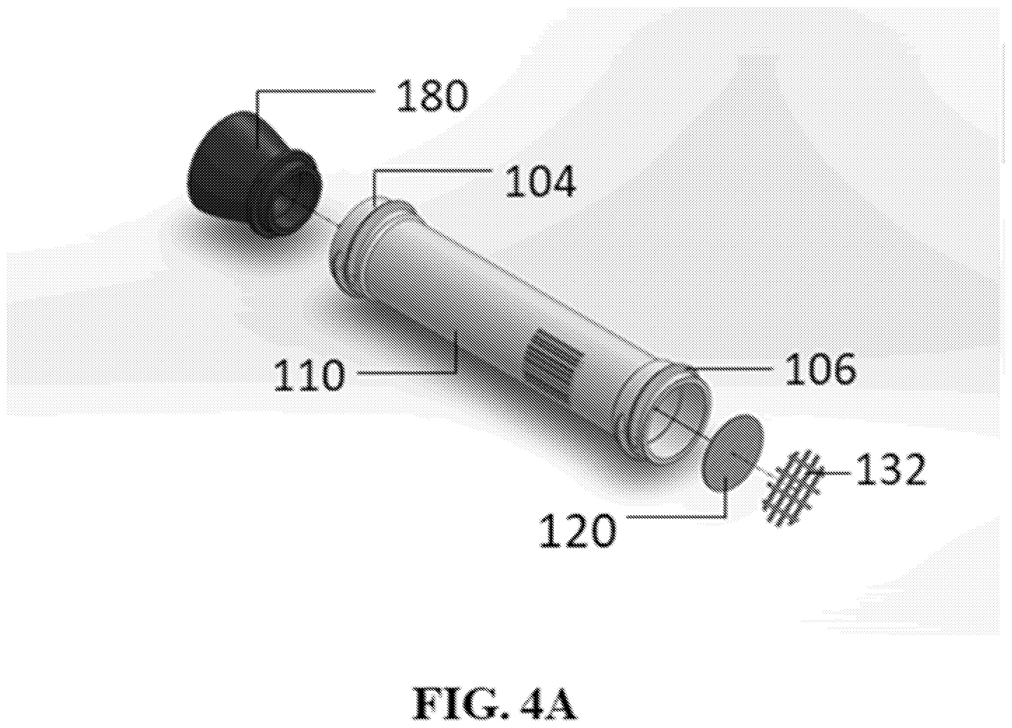

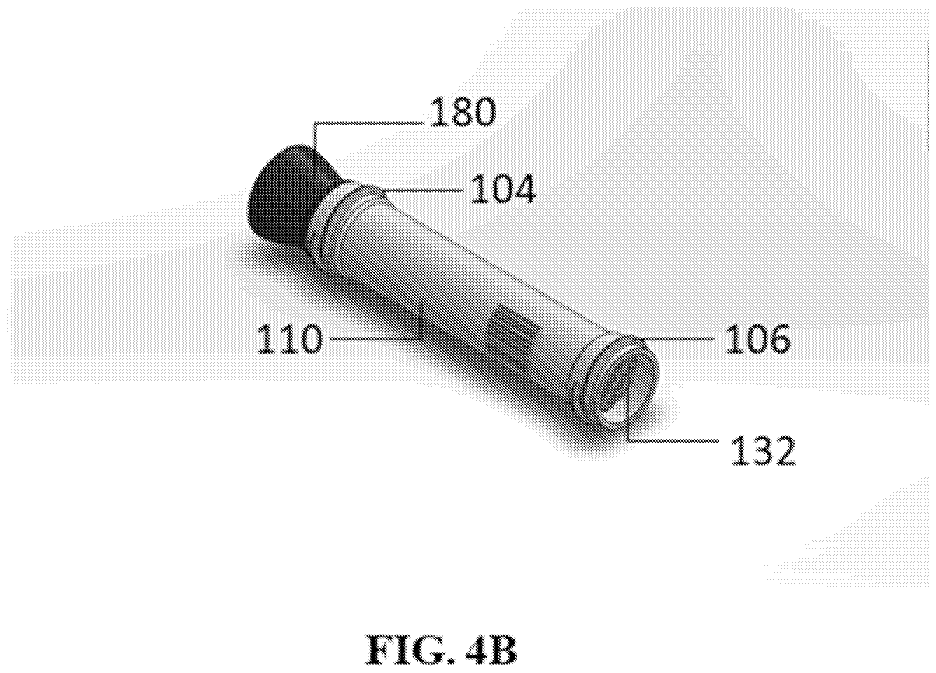

As shown in FIG. 3, in some embodiments, the device 100 further comprises a detector 160 to detect strength of breath and/or loudness of voice by the subject. In some embodiments, the detector 160 may be positioned in the inner space 102 of the conduit 110. In some embodiments, the detector 160 may be positioned between the first opening 104 and the second opening 106 of the conduit 110. The detector 160 may comprise a perforated membrane (not shown). The perforated membrane is configured such that when a subject's breath strength or a subject's voice loudness reaches to a threshold, the perforated membrane could break. In some embodiments, the exhaled air is provided by speaking by the subject. In some embodiments, the device 100 further comprises a blocking structure 170 to prevent backflow, wherein the blocking structure is positioned between the filter 120 and the second opening 106. The blocking structure 170 may be a second filter or a one-way valve. As shown in FIGS. 4A and 4B, in some embodiments, the device 100 may further comprise a mouthpiece 180, wherein the mouthpiece 180 may be attached to the first opening 104 to receive the exhaled air. The mouthpiece can be any shape or size that is suitable for collecting the exhaled air, such as cup-shape. The mouthpiece may be disposable or reusable.

The present disclosure further describes a device for collecting aerosol sample from a subject, comprising a conduit defining an inner space for the passage of exhaled air, wherein the conduit has a first opening for receiving the exhaled air from the subject; a filter positioned in the inner space; and a detector to detect strength of breath, loudness of voice, or both, wherein the detector is positioned in the inner space between the first opening and the filter. In some embodiments, the detector comprises a perforated membrane which is positioned in the inner space. In particular, the perforated membrane is positioned between the opening and the filter.

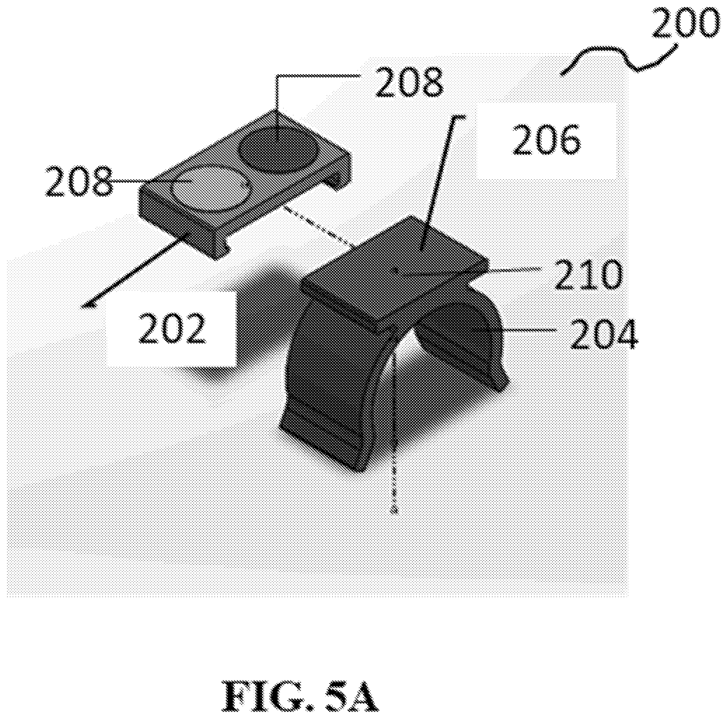

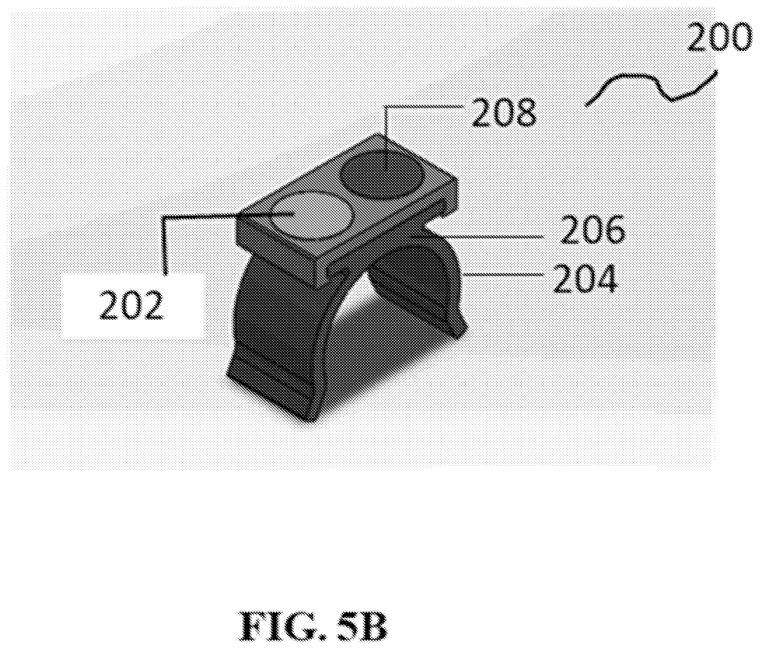

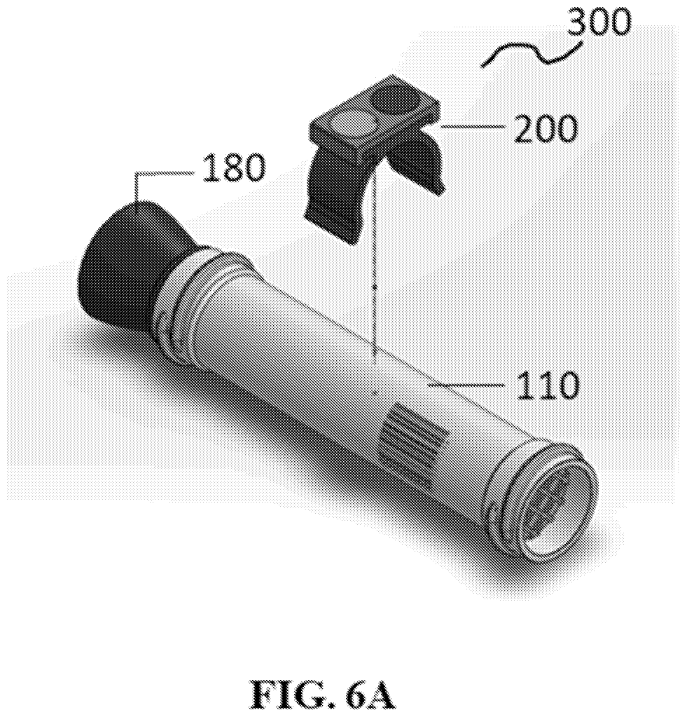

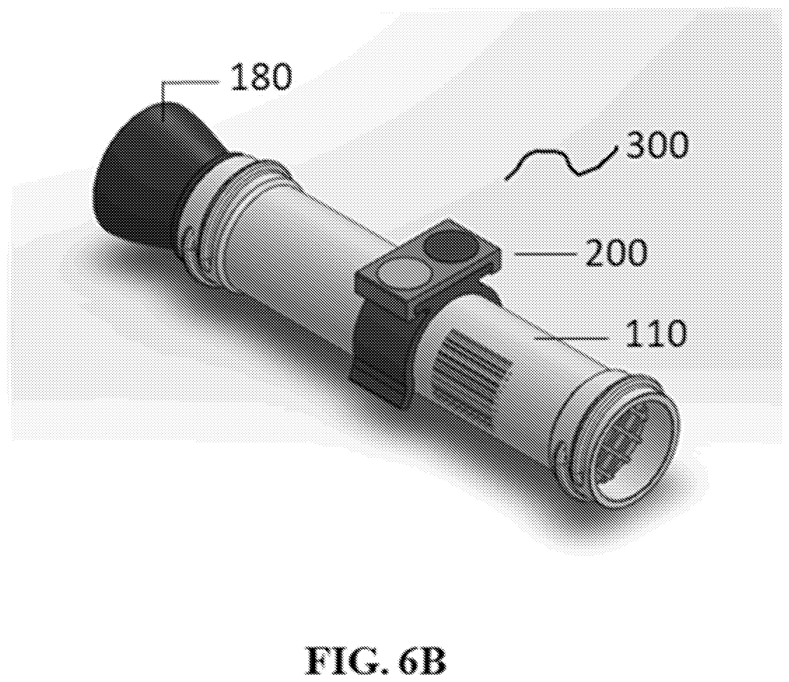

The present disclosure further describes a detecting system for detecting strength of breath and/or loudness of voice of a subject. As shown in FIGS. 5A and 5B, the detecting system 200 comprises a sensor 206 for collecting information regarding the strength of the breath and/or the loudness of the voice of the subject and providing the information to a processor 202, and the processor 202 for processing the information provided by the sensor 206. In some embodiments, the sensor 206 comprises a sound amplifier 210 (e.g., a microphone). In some embodiments, the sound amplifier is a passive amplifier/mechanical amplifier or an active amplifier/electronic amplifier. In some embodiments, the sound amplifier is a sound port. In some embodiments, the sound amplifier is a microphone. In some embodiments, the sensor 206 is fixated on a structure 204 configured to attach to the conduit 110 of the devices as disclosed herein. In some embodiments, the structure 204 is in an arch-shape that fits the conduit 110 of the devices as disclosed herein. For example, as shown in FIGS. 6A and 6B, the structure can be snapped into a position of the conduit of the devices as disclosed herein, that is, the structure can fit quickly into that position. The structure 204 may be placed on any position of the conduit 110. In some embodiments, the structure is placed near the first opening 104 of the conduit. In some embodiments, the structure can be removed from the conduit. In some embodiments, the sensor 206 and the processor 202 are aligned to each other. In some embodiments, the sensor 206, the processor 202, and the structure 204 are attached as one assembly. In some embodiments, the one assembly of the sensor, the processor, and the structure is reusable. In some embodiments, the sensor 206 is reusable. In some embodiments, the processor 202 is reusable. In some embodiments, the structure 204 is reusable. In some embodiments, the sensor 206 and the processor 202 are not physically attached. In some embodiments, the sensor and the processor are connected through a wireless device. For example, the sensor may be paired with the processor by a Bluetooth device (not shown). In some embodiments, the processor 202 comprises at least one indicator 208 for indicating the strength of the breath and/or the loudness of the voice of the subject based on the information provided by the sensor. For example, the indicator may be a light indicator, wherein the light is on when the processor determines that the strength of the breath and/or the loudness of the voice passes a preset threshold. In some embodiments, the indicator can indicate if the subject speaks the preset words or phrases. In some embodiments, the processor comprises two indicators, wherein one indicator indicates pass and the other indicator indicates fail. For example, the processor comprises two light indicators with different colors (for example, red and green, as shown in FIGS. 5A and 5B and FIGS. 6A and 6B). When the strength of the breath and/or the loudness of the voice passes the preset threshold, or when the subject speaks the preset words or phrases, the green light indicator will be on to indicate pass, otherwise the red light indicator will be on to indicate fail.

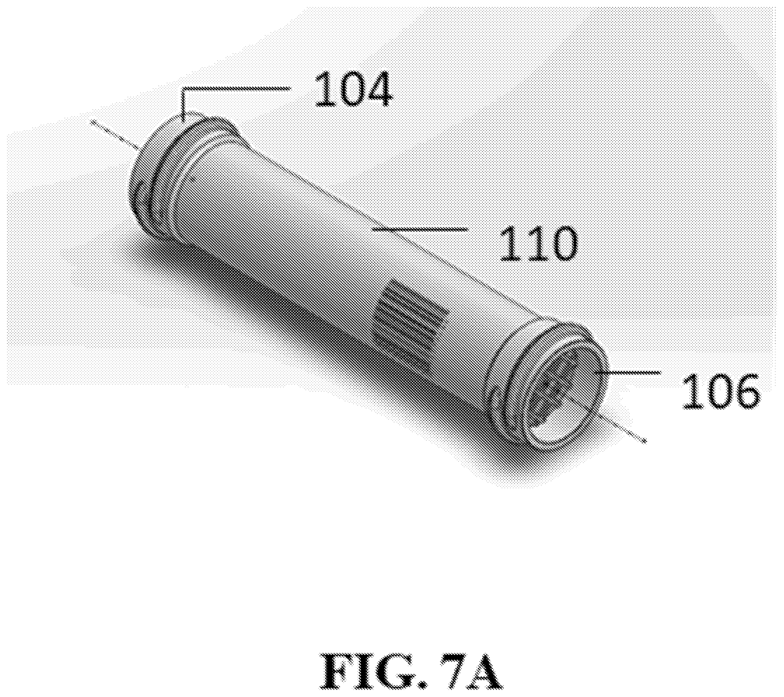

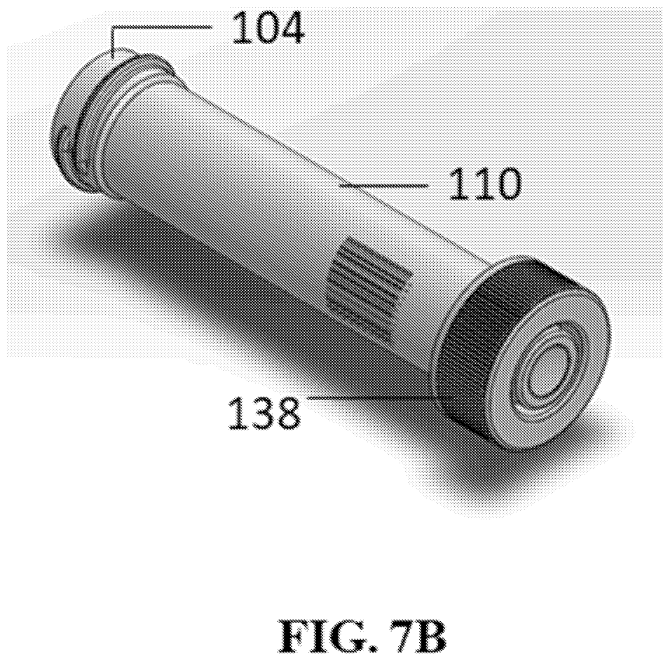

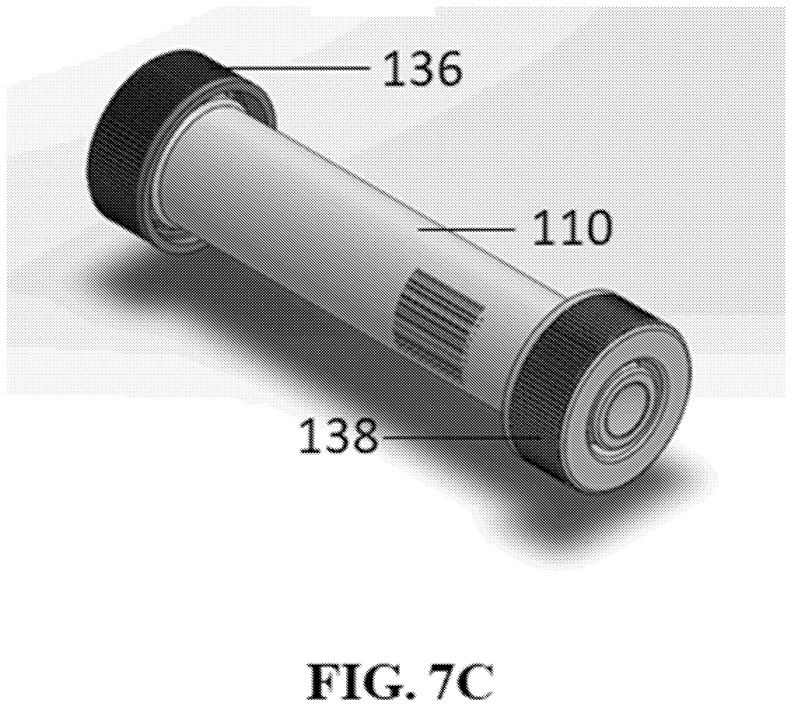

As shown in FIGS. 6A and 6B, the present disclosure further describes a system 300 for collecting aerosol sample from a subject, comprising a conduit 110 defining an inner space for the passage of exhaled air, wherein the conduit 110 has an opening for receiving exhaled air from the subject; and a filter for collecting the aerosol sample from the exhaled air, and the detecting system 200 disclosed herein to detect the strength of breath, loudness of voice, or both. In some embodiments, the system may further comprise a mouthpiece 180, wherein the mouthpiece is attached to the first opening to receive the exhaled air. After the subject uses (e.g., breathes or speaks into) the conduit 110 of the system, as shown in FIG. 7A, the mouthpiece 180 and/or the detecting system 200 may be removed from the conduit 110. A cap 138 may be installed on one opening of the conduit that is near the filter (e.g., the second opening) as shown in FIG. 7B. By installing the cap 138 on the second opening, the device is ready for solvent addition. Media may be added to elute the samples on the filter. Another cap 136 may be installed on the other opening of the conduit (e.g., the first opening) as shown in FIG. 7C. By installing both caps on both openings of the conduit, the device is ready for storage.

A device configured to collect speech-generated droplets from a subject is disclosed herein, comprising a conduit defining an inner space for the passage of exhaled air comprising the speech-generated droplets, wherein the conduit has an opening for receiving the exhaled air from the subject; and a filter for collecting the speech-generated droplets from the exhaled air, wherein the filter is positioned in the inner space, wherein the speech-generated droplets are produced from speaking by the subject. In some embodiments, the exhaled air is provided by speaking by the subject. In some embodiments, the device further comprising a detector to detect the strength of breath, loudness of voice, or both. In some embodiments, the detector is positioned in the inner space of the conduit. In some embodiments, the detector comprises a perforated membrane. In some embodiments, the detector is the detecting system disclosed herein.

In some embodiments, the filter exhibits a low airflow resistance. In some embodiments, the filter exhibits an airflow resistance of less than about 1.5 cm H₂O/L/sec at flow rates of about 14 L/sec. In some embodiments, the filter is electrostatically charged.

In some embodiments, the device further comprises a long axis and a second opening for expelling at least a portion of the exhaled air, wherein the filter is positioned between the first opening and the second opening. In some embodiments, the device further comprises a filter support, wherein the filter support is positioned between the first opening and the second opening and is substantially perpendicular to the long axis, and wherein the filter is secured to the filter support. In some embodiments, the filter support comprises a filter web. In some embodiments, the filter web comprises one or more opening. The opening can be any shape. In some embodiments, the filter web comprises resin. In some embodiments, the filter web comprises polypropylene or polyethylene terephthalate. In some embodiments, the conduit comprises polypropylene or polyethylene terephthalate. In some embodiments, the filter web is made from the same resin as the conduit. In some embodiments, the filter web is made from the same polypropylene or polyethylene terephthalate as the conduit. In some embodiments, the filter support further comprises a shoulder. In some embodiments, the filter is secured between the shoulder and the filter web. In some embodiments, the filter support is permanently secured to the conduit. In some embodiments, the filter support is sonically welded to the conduit. In some embodiments, the cross-sectional area of the inner space of the conduit between the first opening and the second opening is constant. In some embodiments, the length of the conduit between the first opening and the second opening is about 100 to about 200 millimeters. In some embodiments, the diameter of the conduit is about 10 to about 20 millimeters. In some embodiments, the diameter of the conduit is about 16 millimeters. In some embodiments, the conduit fits the requirements of an industry standard 10 mL centrifuge tube, including diameters and wall thickness requirements. In some embodiments, the conduit volumetrically compatible with current CDC/FDA guidelines for COVID-19 sample collection/storage in the field by trained personnel. In some embodiments, the conduit has a 20 mL total volume. In some embodiments, the conduit has a 10 mL total volume.

In some embodiments, the device further comprises a first removable cap, wherein the first removable cap can secure the first opening. In some embodiments, the device further comprises a second removable cap, wherein the second removable cap can secure the second opening. In some embodiments, the first opening has a threaded surrounding wall that mates with a threaded surrounding wall of the first removable cap. In some embodiments, the second opening has a threaded surrounding wall that mates with a threaded surrounding wall of the second removable cap. In some embodiments, the first removable cap has an indicator indicating that the cap is the first removable cap. In some embodiments, the second removable cap has an indicator indicating that the cap is the second removable cap. In some embodiments, the indicator may be of color, symbol, or shapes. In some embodiments, the first removable cap and the second removable cap comprise different colors. In some embodiments, the device further comprises a removable film, wherein the removable film covers at least a portion of an outside wall of the conduit. In some embodiments, the removable film covers the whole outside wall of the conduit. The removable film may be a tear-away film. The removable film may be used to prevent against contamination from human contact (e.g., patient's lips and/or fingers) during sampling. In some embodiments, the device further comprises a mouthpiece, wherein the mouthpiece is attached to the first opening to receive the exhaled air.

### Electrically Charged Filter

An electrically charged filter is a filter having dielectric properties or capable of being enhanced to have dielectric properties. The filter may comprise a fiber-based material having a fibrous matrix structure. The filter may comprise a sponge-like material having an open cell matrix structure. The material may be flexible or inflexible. In some embodiments, the filter comprises a nonwoven fabric. The nonwoven fabric may be a manufactured sheet, mat, web or batt of directionally or randomly oriented fibers bonded by friction or adhesion. In some embodiments, the filter may comprise a nonwoven textile of varying fluffiness, comprising polymer fiber. The polymer may be nylon, polyethylene, polypropylene, polyester, etc. or any other polymer suitable for a filter substrate. Additionally, the substrate can be made of materials other than polymer fiber.

### Low Airflow Resistance Filter

In some instances, the filter of the device exhibits a low airflow resistance. In some embodiments, the filter exhibits an airflow resistance of less than 1.5 cm H₂O/L/sec at flow rates of 14 L/sec. In some embodiments, the filter exhibits an airflow resistance of less than about 1.0, less than about 0.9, less than about 0.8, less than about 0.7, less than about 0.6, less than about 0.5, less than about 0.4, less than about 0.3, less than about 0.2, or less than about 0.1 cm H₂O/L/sec at flow rates of 14 L/sec. The filter may comprise a nonwoven synthetic fiber or a nanofiber. The filter may comprise a material that is non-chemically reactive with the aerosol sample. The material may be polypropylene, polyester, or nylon. In some instances, the filter is also electrostatically charged. The filter may be hydrophobic. In some instances, the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% bacterial filtration efficiency. In some instances, the filter exhibits at least about 99%, at least about 99.1%, at least about 99.2%, at least about 99.3%, at least about 99.4%, at least about 99.5%, at least about 99.6%, at least about 99.7%, at least about 99.8%, or at least about 99.9% bacterial filtration efficiency. In some instances, the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency. In some instances, the filter exhibits at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency.

### Methods to Collect Aerosol Sample

The present disclosure describes a method for collecting an aerosol sample from a subject, comprising obtaining or having obtained any one of the devices as described herein; and receiving exhaled air from the subject into the inner space through the first opening. In some embodiments, the exhaled air is provided by speaking by the subject. In some embodiments, the subject speaks repeatedly a phrase with an average loudness of about 59 dB_{B} at a distance of about 30 centimeters. In some embodiments, the subject speaks repeatedly a phrase with a loudness of about 85 dB_{B} or less at a distance of about 30 centimeters. In some embodiments, the aerosol sample comprises speech-generated droplets. The speech-generated droplets may have a size from 1 μm to 150 μm. The speech-generated droplets may have a size from about 10 μm to about 100 μm. In some embodiments, the speech-generated droplets may have a size from about 20 μm to about 90 μm. In some embodiments, the speech-generated droplets may have a size from about 30 μm to about 80 μm. In some embodiments, the speech-generated droplets may have a size from about 40 μm to about 70 μm. In some embodiments, the speech-generated droplets may have a size from about 50 um to about 60 μm. In some embodiments, the method further comprises measuring the loudness of the speaking. A detector may be used to measure the loudness of the speaking. In some embodiments, the method further comprises securing a second cap to the second opening. In some embodiments, the method further comprises adding a medium to the inner space of the device, whereby the filter is immersed in the medium. In some embodiments, the method further comprises securing the first cap to the first opening. In some embodiments, the method further comprises detecting a molecule or pathogen from the filter. In some embodiments, the molecule or pathogen is detected by color, an optical signal, an electronic signal, a chemical signal, or by artificial intelligence. In some embodiments, the molecule or pathogen is detected by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy.

The present disclosure describes a method for collecting an aerosol sample from a subject, comprising providing a device comprising a filter for collecting aerosol sample from a subject and receiving exhaled air from the subject, wherein the exhaled air is provided by speaking by the subject. In some embodiments, the method further comprises detecting the loudness of the speaking. A detector may be used to measure the loudness of the speaking. In some embodiments, the subject speaks repeatedly a phrase with an average loudness of about 59 dB_{B} at a distance of about 30 centimeters. In some embodiments, the subject speaks repeatedly a phrase with a loudness of about 85 dB_{B} or less at a distance of about 30 centimeters. In some embodiments, the aerosol sample comprises speech-generated droplets. The speech-generated droplets may have a size from about 1 μm to about 150 μm. The speech-generated droplets may have a size from about 10 μm to about 100 μm. In some embodiments, the speech-generated droplets may have a size from about 20 μm to about 90 μm. In some embodiments, the speech-generated droplets may have a size from about 30 μm to about 80 μm. In some embodiments, the speech-generated droplets may have a size from about 40 μm to about 70 μm. In some embodiments, the speech-generated droplets may have a size from about 50 μm to about 60 μm. In some embodiments, the filter is electrostatically charged. In some embodiments, the filter exhibits a low airflow resistance.

In some embodiments, the method further comprises adding a medium to the device such that the filter is immersed in the medium. In some embodiments, the method further comprises detecting a molecule or pathogen from the filter. In some instances, the molecule or pathogen is detected by color, optical, electronic, chemical or artificial intelligence. For example, the molecule or pathogen may be detected by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy. In some embodiments, the molecule or pathogen is a bacterium, a virus or a viral particle, In some embodiments, the virus is SARS-COV-2, a variant or a mutant thereof.

### Kits

The present disclosure describes a kit for collecting an aerosol sample from a subject, comprising any one of the devices as described herein, and instructions for collecting an aerosol sample. In some embodiments, the kit further comprises a resealable bag.

In some embodiments, the kit further comprises a medium. In some embodiments, the medium has a volume of from about 1 milliliter to about 20 milliliters. In some embodiments, the medium has a volume of from about 5 milliliters to about 15 milliliters. In some embodiments, the medium has a volume of from about 8 milliliters to about 12 milliliters. In some embodiments, the medium has a volume of about 10 milliliters. In some embodiments, the medium has a volume of from about 1 milliliter to about 10 milliliters. In some embodiments, the medium has a volume of from about 2 milliliter to about 9 milliliters. In some embodiments, the medium has a volume of from about 3 milliliter to about 8 milliliters. In some embodiments, the medium has a volume of from about 4 milliliter to about 7 milliliters. In some embodiments, the medium has a volume of from about 5 milliliter to about 6 milliliters. In some embodiments, the medium has a volume of from about 1 milliliter to about 5 milliliters. In some embodiments, the medium has a volume of from about 2 milliliter to about 4 milliliters. In some embodiments, the medium has a volume of about 3 milliliters. In some embodiments, the medium comprises a proteinaceous solution. In some embodiments, the proteinaceous solution comprises beef extract. The beef extract may have a concentration from about 1.5% to about 3.0%. In some embodiments, the beef extract comprises veal infusion broth or fetal bovine serum. In some embodiments, the medium further comprises an antibiotic. In some embodiments, the antibiotic is gentamicin sulfate. In some embodiments, the medium further comprises an antifungal, such as amphotericin B. In some embodiments, the medium further comprises a salt-based solution, such as Hank's Balanced Salt Solution (HBSS). The salt-based solution may further comprise calcium and magnesium ions. In some embodiments, the medium further comprises an eluting solution. The eluting solution may comprise glycine. The eluting solution may further comprise at least one amphoteric amino acid. The eluting solution may further comprise a sodium polyphosphate solution. In some embodiments, the kit further comprises a unique barcode label for traceability. In some embodiments, the unique barcode label is attached to the device. In some embodiments, the kit further comprises the detecting system disclosed herein.

### Embodiments

Some embodiments include one or more of the following:

Embodiment 1: A device for collecting an aerosol sample from a subject, the device comprising: (a) a conduit defining an inner space for passage of exhaled air, wherein the conduit comprises: (i) a first opening for receiving the exhaled air from the subject; (ii) a second opening for expelling at least a portion of the exhaled air; and (iii) a long axis; and (b) a filter for collecting the aerosol sample from the exhaled air, wherein the filter is positioned between the first opening and the second opening and is substantially perpendicular to the long axis, and wherein the filter is an electrostatically charged filter.

Embodiment 2: A device for collecting an aerosol sample from a subject, the device comprising: (a) a conduit defining an inner space for passage of exhaled air, wherein the conduit comprises: (i) a first opening for receiving the exhaled air from the subject; (ii) a second opening for expelling at least a portion of the exhaled air; and (iii) a long axis; and (b) a filter for collecting the aerosol sample from the exhaled air, wherein the filter is positioned between the first opening and the second opening and is substantially perpendicular to the long axis, and wherein the filter exhibits a low airflow resistance.

Embodiment 3: The device of embodiment 1, wherein the filter exhibits a low airflow resistance.

Embodiment 4: The device of embodiment 2 or 3, wherein the filter exhibits an airflow resistance of less than about 1.5 cm H₂O/L/sec at flow rates of about 14 L/sec.

Embodiment 5: The device of any one of embodiments 1-4, further comprising a filter support, wherein the filter support is positioned between the first opening and the second opening and is substantially perpendicular to the long axis, and wherein the filter is secured to the filter support.

Embodiment 6: The device of embodiment 5, wherein the filter support comprises a filter web.

Embodiment 7: The device of embodiment 6, wherein the filter web comprises one or more opening.

Embodiment 8: The device of embodiment 6 or 7, wherein the filter web comprises resin.

Embodiment 9: The device of any one of embodiments 6-8, wherein the filter web comprises polypropylene or polyethylene terephthalate.

Embodiment 10: The device of any one of embodiments 1-9, wherein the conduit comprises resin.

Embodiment 11: The device of any one of embodiments 1-10, wherein the conduit comprises polypropylene or polyethylene terephthalate.

Embodiment 12: The device of any one of embodiments 5-11, wherein the filter support further comprises a shoulder.

Embodiment 13: The device of embodiment 12, wherein the filter is secured between the shoulder and the filter web.

Embodiment 14: The device of any one of embodiments 5-13, wherein the filter support is permanently secured to the conduit.

Embodiment 15: The device of embodiment 14, wherein the filter support is sonically welded to the conduit.

Embodiment 16: The device of any one of embodiments 1-15, wherein a cross-sectional area of the inner space between the first opening and the second opening is constant.

Embodiment 17: The device of any one of embodiments 1-16, wherein the length of the conduit between the first opening and the second opening is about 100 to about 150 millimeters.

Embodiment 18: The device of any one of embodiments 1-17, wherein the diameter of the conduit is about 10 to 20 about millimeters.

Embodiment 19: The device of embodiment 18, wherein the diameter of the conduit is about 16 millimeters.

Embodiment 20: The device of any one of embodiments 1-19, further comprising a first removable cap, wherein the first removable cap is configured to secure the first opening.

Embodiment 21: The device of any one of embodiments 1-20, further comprising a second removable cap, wherein the second removable cap is configured to secure the second opening.

Embodiment 22: The device of embodiment 20 or 21, wherein the first opening has a threaded surrounding wall that mates with a threaded surrounding wall of the first removable cap.

Embodiment 23: The device of any one of embodiments 20-22, wherein the second opening has a threaded surrounding wall that mates with a threaded surrounding wall of the second removable cap.

Embodiment 24: The device of any one of embodiments 21-23, wherein the first removable cap and the second removable cap comprise different colors.

Embodiment 25: The device of any one of embodiments 1-24, further comprising a removable film, wherein the removable film covers at least a portion of an outside wall of the conduit.

Embodiment 26: The device of any one of embodiments 1-25, wherein the filter exhibits an airflow resistance of less than about 1.0, less than about 0.9, less than about 0.8, less than about 0.7, less than about 0.6, less than about 0.5, less than about 0.4, less than about 0.3, less than about 0.2, or less than about 0.1 cm H₂O/L/sec at flow rates of about 14 L/sec.

Embodiment 27: The device of any one of embodiments 1-26, wherein the filter comprises a non-woven synthetic fiber or a nanofiber.

Embodiment 28: The device of any one of embodiments 1-27, wherein the filter comprises a material that is non-chemically reactive with the aerosol sample.

Embodiment 29: The device of embodiment 28, wherein the material is polypropylene, polyester, or nylon.

Embodiment 30: The device of any one of embodiments 1-29, wherein the filter is hydrophobic.

Embodiment 31: The device of any one of embodiments 1-30, wherein the filter exhibits at least about 95%, at least about 96%, at least about 97%, at least about 98%, at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency.

Embodiment 32: The device of any one of embodiments 1-31, wherein the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency.

Embodiment 33: The device of any one of embodiments 1-32, wherein the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency.

Embodiment 34: The device of any one of embodiments 1-33, wherein the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% viral filtration efficiency.

Embodiment 35: The device of any one of embodiments 1-34, wherein the filter is positioned near the second opening.

Embodiment 36: The device of any one of embodiments 1-35, wherein the filter is positioned such that the filter can be immersed in a medium.

Embodiment 37: The device of embodiment 36, wherein the medium has a volume of less than about 20 milliliters.

Embodiment 38: The device of embodiment 35, wherein the medium has a volume of less than 5 milliliters.

Embodiment 39: The device of embodiment 36, wherein the medium has a volume of about 3 milliliters.

Embodiment 40: The device of any one of embodiments 1-39, wherein the filter comprises a sensor configured to detect a molecule or a pathogen.

Embodiment 41: The device of embodiment 38, wherein the sensor detects the molecule or the pathogen by color, an optical signal, an electronic signal, a chemical signal, or by artificial intelligence.

Embodiment 42: The device of embodiment 38, wherein the sensor detects the molecule or the pathogen by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy.

Embodiment 43: The device of any one of embodiments 40-42, wherein the sensor is configured to detect a bacterium, a virus, or a viral particle.

Embodiment 44: The device of embodiment 43, wherein the virus or viral particle is severe acute respiratory syndrome coronavirus-2 (SARS-CoV-2), a variant thereof, or a mutant thereof.

Embodiment 45: The device of any one of embodiments 1-45, further comprising a detector to detect strength of breath of the subject, loudness of voice of the subject, or both.

Embodiment 46: The device of embodiment 45, wherein the detector is positioned between the first opening and the second opening.

Embodiment 47: The device of embodiment 46, wherein the detector comprises a perforated membrane.

Embodiment 48: The device of embodiment 45, wherein the detector is attached to an outside wall of the conduit.

Embodiment 49: The device of embodiment 48, wherein the detector is a detecting system that comprises (a) a sensor for collecting information regarding the strength of the breath of the subject, the loudness of the voice of the subject, or both, and providing the information to a processor; (b) the processor for processing the information provided by the sensor; and (c) a structure configured to allow the sensor to attach to the conduit.

Embodiment 50: The device of any one of embodiments 1-49, wherein the device is configured to capture speech-generated droplets provided by a subject while speaking.

Embodiment 51: The device of any one of embodiments 1-50, further comprising a blocking structure to prevent backflow, wherein the blocking structure is positioned between the filter and the second opening.

Embodiment 52: The device of embodiment 51, wherein the blocking structure is a second filter or a one-way valve.

Embodiment 53: The device of any one of embodiments 1-52, further comprising a mouthpiece, wherein the mouthpiece is attached to the first opening to receive the exhaled air.

Embodiment 54: A device for collecting an aerosol sample from a subject, comprising: (a) a conduit defining an inner space for passage of exhaled air, wherein the conduit has a first opening for receiving the exhaled air from the subject; (b) a filter positioned in the inner space; and (c) a detector to detect strength of breath, loudness of voice, or both, wherein the detector is positioned in the inner space.

Embodiment 55: The device of embodiment 54, wherein the detector is positioned between the first opening and the filter.

Embodiment 56: A device configured to collect speech-generated droplets from a subject, comprising: (a) a conduit defining an inner space for passage of exhaled air comprising the speech-generated droplets, wherein the conduit has an opening for receiving the exhaled air from the subject; and (b) a filter for collecting the speech-generated droplets from the exhaled air, wherein the filter is positioned in the inner space, wherein the speech-generated droplets are produced from speaking by the subject.

Embodiment 57: The device of embodiment 56, wherein the exhaled air is provided by speaking by the subject.

Embodiment 58: The device of embodiment 57, further comprising a detector to detect strength of breath, loudness of voice, or both.

Embodiment 59: The device of embodiment 58, wherein the detector is positioned in the inner space.

Embodiment 60: The device of embodiment 58 or 59, wherein the detector comprises a perforated membrane.

Embodiment 61: The device of embodiment 58, wherein the detector is attached to an outside wall of the conduit.

Embodiment 62: The device of embodiment 61, wherein the detector is a detecting system that comprises (a) a sensor for collecting information regarding the strength of the breath of the subject, the loudness of the voice of the subject, or both, and providing the information to a processor; (b) the processor for processing the information provided by the sensor; and (c) a structure configured to allow the sensor to attach to the conduit of the devices.

Embodiment 63: The device of any one of embodiments 56-62, wherein the filter exhibits a low airflow resistance.

Embodiment 64: The device of embodiment 63, wherein the filter exhibits an airflow resistance of less than about 1.5 cm H₂O/L/sec at flow rates of about 14 L/sec.

Embodiment 65: The device of any one of embodiments 56-64, wherein the filter is electrostatically charged.

Embodiment 66: The device of any one of embodiments 56-65, further comprising a long axis and a second opening for expelling at least a portion of the exhaled air, wherein the filter is positioned between the first opening and the second opening.

Embodiment 67: The device of any one of embodiments 56-66, further comprising a filter support, wherein the filter support is positioned between the first opening and the second opening and is substantially perpendicular to the long axis, and wherein the filter is secured to the filter support.

Embodiment 68: The device of embodiment 67, wherein the filter support comprises a filter web.

Embodiment 69: The device of embodiment 68, wherein the filter web comprises one or more opening.

Embodiment 70: The device of embodiment 68 or 69, wherein the filter web comprises resin.

Embodiment 71: The device of any one of embodiments 68-70, wherein the filter web comprises polypropylene or polyethylene terephthalate.

Embodiment 72: The device of any one of embodiments 56-71, wherein the conduit comprises resin.

Embodiment 73: The device of any one of embodiments 56-72, wherein the conduit comprises polypropylene or polyethylene terephthalate.

Embodiment 74: The device of any one of embodiments 67-73, wherein the filter support further comprises a shoulder.

Embodiment 75: The device of embodiment 74, wherein the filter is secured between the shoulder and the filter web.

Embodiment 76: The device of any one of embodiments 67-75, wherein the filter support is permanently secured to the conduit.

Embodiment 77: The device of embodiment 76, wherein the filter support is sonically welded to the conduit.

Embodiment 78: The device of any one of embodiments 56-77, wherein a cross-sectional area of the inner space between the first opening and the second opening is constant.

Embodiment 79: The device of any one of embodiments 56-78, wherein the length of the conduit between the first opening and the second opening is about 100 to about 150 millimeters.

Embodiment 80: The device of any one of embodiments 56-79, wherein the diameter of the conduit is about 10 to about 20 millimeters.

Embodiment 81: The device of any one of embodiments 56-80, further comprising a first removable cap, wherein the first removable cap can secure the first opening.

Embodiment 82: The device of any one of embodiments 56-81, further comprising a second removable cap, wherein the second removable cap can secure the second opening.

Embodiment 83: The device of embodiment 81 or 82, wherein the first opening has a threaded surrounding wall that mates with a threaded surrounding wall of the first removable cap.

Embodiment 84: The device of embodiment 82 or 83, wherein the second opening has a threaded surrounding wall that mates with a threaded surrounding wall of the second removable cap.

Embodiment 85: The device of any one of embodiments 81-84, wherein the first removable cap and the second removable cap comprise different colors.

Embodiment 86: The device of any one of embodiments 56-85, further comprising a removable film, wherein the removable film covers at least a portion of an outside wall of the conduit.

Embodiment 87: The device of any one of embodiments 56-86, wherein the filter exhibits an airflow resistance of less than about 1.0, less than about 0.9, less than about 0.8, less than about 0.7, less than about 0.6, less than about 0.5, less than about 0.4, less than about 0.3, less than about 0.2, or less than about 0.1 cm H₂O/L/sec at flow rates of about 14 L/sec.

Embodiment 88: The device of any one of embodiments 56-87, wherein the filter comprises a non-woven synthetic fiber or a nanofiber.

Embodiment 89: The device of any one of embodiments 56-88, wherein the filter comprises a material that is non-chemically reactive with the aerosol sample.

Embodiment 90: The device of embodiment 89, wherein the material is polypropylene, polyester, or nylon.

Embodiment 91: The device of any one of embodiments 56-90, wherein the filter is hydrophobic.

Embodiment 92: The device of any one of embodiments 56-91, wherein the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% bacterial filtration efficiency.

Embodiment 93: The device of any one of embodiments 56-92, wherein the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% bacterial filtration efficiency.

Embodiment 94: The device of any one of embodiments 56-93, wherein the filter exhibits at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9% viral filtration efficiency.

Embodiment 95: The device of any one of embodiments 56-94, wherein the filter exhibits at least about 99%, at least about 99.1%, at least about 99.2%, at least about 99.3%, at least about 99.4%. at least about 99.5%, at least about 99.6%, at least about 99.7%, at least about 99.8%, or at least about 99.9% viral filtration efficiency.

Embodiment 96: The device of any one of embodiments 56-95, wherein the filter is positioned near the second opening.

Embodiment 97: The device of any one of embodiments 56-96, wherein the filter comprises a sensor that detects a molecule or pathogen.

Embodiment 98: The device of embodiment 97, wherein the sensor detects the molecule or pathogen by a color signal, an optical signal, an electronic signal, a chemical signal, or by artificial intelligence.

Embodiment 99: The device of embodiment 98, wherein the sensor detects the molecule or pathogen by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy.

Embodiment 100: The device of any one of embodiments 97-99, wherein the sensor detects a bacterium, a virus, or a viral particle.

Embodiment 101: The device of any one of embodiments 56-100, further comprising a blocking structure to prevent backflow, wherein the blocking structure is positioned between the filter and the second opening.

Embodiment 102: The device of embodiment 101, wherein the blocking structure is a second filter or a one-way valve.

Embodiment 103: The device of any one of embodiments 56-102, further comprising a mouthpiece, wherein the mouthpiece is attached to the first opening to receive the exhaled air.

Embodiment 104: A method for collecting an aerosol sample from a subject, comprising obtaining or having obtained a device of any one of the preceding claims; and receiving exhaled air from the subject into the inner space through the first opening of the device.

Embodiment 105: The method of embodiment 104, wherein the exhaled air is provided by speaking by the subject.

Embodiment 106: The method of embodiment 105, wherein the speaking comprises repeatedly speaking a phrase with an average loudness of about 59 dBB at a distance of about 30 centimeters.

Embodiment 107: The method of embodiment 106, wherein the speech of the subject comprises repeatedly speaking a phrase with a loudness of about 85 dBB or less at a distance of about 30 centimeters.

Embodiment 108: The method of any one of embodiments 104-107, wherein the aerosol sample comprises speech-generated droplets.

Embodiment 109: The method of embodiment 108, wherein the speech-generated droplets have a size from about 1 μm to about 150 μm.

Embodiment 110: The method of embodiment 109, wherein the speech-generated droplets have a size from about 10 μm to about 100 μm.

Embodiment 111: The method of any one of embodiments 104-110, further comprising measuring loudness of the speaking.

Embodiment 112: The method of any one of embodiments 104-111, further comprising securing a second cap to a second opening.

Embodiment 113: The method of embodiment 112, further comprising adding a medium to the inner space of the device, whereby the filter is immersed in the medium.

Embodiment 114: The method of any one of embodiments 104-113, further comprising securing a first cap to a first opening.

Embodiment 115: The method of any one of embodiments 104-114, further comprising detecting a molecule or pathogen from the filter.

Embodiment 116: The method of embodiment 115, wherein the molecule or pathogen is detected by color, an optical signal, an electronic signal, a chemical signal, or by artificial intelligence.

Embodiment 117: The method of embodiment 115, wherein the molecule or pathogen is detected by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy.

Embodiment 118: A method for collecting an aerosol sample from a subject, comprising providing a device comprising a filter for collecting the aerosol sample from the subject; and receiving exhaled air from the subject, wherein the exhaled air is provided by speaking by the subject.

Embodiment 119: The method of embodiment 118, further comprising detecting loudness of the speaking.

Embodiment 120: The method of embodiment 119, wherein the speaking comprises repeatedly speaking a phrase with an average loudness of about 59 dBB at a distance of about 30 centimeters.

Embodiment 121: The method of embodiment 120, wherein the speaking comprises repeatedly speaking a phrase with a loudness of about 85 dBB or less at a distance of about 30 centimeters.

Embodiment 122: The method of any one of embodiments 118-121, wherein the aerosol sample comprises speech-generated droplets.

Embodiment 123: The method of embodiment 122, wherein the speech-generated droplets have a size from about 1 μm to about 150 μm.

Embodiment 124: The method of embodiment 123, wherein the speech-generated droplets have a size from about 10 μm to about 100 μm.

Embodiment 125: The method of any one of embodiments 118-124, wherein the filter is electrostatically charged.

Embodiment 126: The method of any one of embodiments 118-125, wherein the filter exhibits a low airflow resistance.

Embodiment 127: The method of any one of embodiments 118-126, further comprising adding a medium to the device such that the filter is immersed in the medium.

Embodiment 128: The method of any one of embodiments 118-127, further comprising detecting a molecule or pathogen from the filter.

Embodiment 129: The method of embodiment 128, wherein the molecule or pathogen is detected by a color signal, an optical signal, an electronic signal, a chemical signal, or by artificial intelligence.

Embodiment 130: The method of embodiment 128, wherein the molecule or pathogen is detected by enzyme-linked immunosorbent assay (ELISA), immunofluorescence (IF), polymerase chain reaction (PCR), reverse transcription recombinase PCR (RT-PCR), real-time quantitative RT-PCR (qRT-PCR), nucleic acid sequence-based amplification (NASBA), RNA-seq, in situ hybridization, nanoparticles, or Raman spectroscopy.

Embodiment 131: The method of any one of embodiments 128-130, wherein the molecule or pathogen is a bacterium, a virus, or a viral particle.

Embodiment 132: The method of embodiment 131, wherein the virus or viral particle is severe acute respiratory syndrome coronavirus-2 (SARS-CoV-2), a variant thereof, or a mutant thereof.

Embodiment 133: A detecting system for detecting strength of breath of a subject, loudness of voice of a subject, or both, the detecting system comprising: (a) a sensor for collecting information regarding the strength of the breath of the subject, the loudness of the voice of the subject, or both, and providing the information to a processor; (b) the processor for processing the information provided by the sensor; and (c) a structure configured to allow the sensor to attach to the conduit of any one of the devices of embodiments 1-103.

Embodiment 134: The detecting system of embodiment 133, wherein the sensor comprises a sound amplifier.

Embodiment 135: The detecting system of embodiment 133, wherein the structure is in an arch-shape that fits the conduit of any one of the devices of embodiments 1-103.

Embodiment 136: The detecting system of any one of embodiments 133-135, wherein the processor comprises at least one indicator for indicating the strength of the breath of the subject, the loudness of the voice of the subject, or both, based on the information provided by the sensor.

Embodiment 137: A system for collecting an aerosol sample from a subject, the system comprising: (a) a conduit defining an inner space for passage of exhaled air, wherein the conduit has a first opening for receiving the exhaled air from the subject; (b) a filter positioned in the inner space; and (c) the detecting system of any one of embodiments 133-136.

Embodiment 138: A kit for collecting an aerosol sample from a subject, the kit comprising a device of any one of embodiments 1-103; and instructions for collecting the aerosol sample.

Embodiment 139: The kit of embodiment 138, further comprising a resealable bag.

Embodiment 140: The kit of embodiment 138 or 139, further comprising a medium.

Embodiment 141: The kit of embodiment 140, wherein the medium has a volume of at least about 3 milliliters.

Embodiment 142: The kit of embodiment 140 or 141, wherein the medium comprises a proteinaceous solution.

Embodiment 143: The kit of embodiment 142, wherein the proteinaceous solution comprises beef extract.

Embodiment 144: The kit of embodiment 143, wherein the beef extract has a concentration from about 1.5% to about 3.0%.

Embodiment 145: The kit of embodiment 143 or 144, wherein the beef extract comprises veal infusion broth or fetal bovine serum.

Embodiment 146: The kit of any one of embodiments 140-145, wherein the medium further comprises an antibiotic.

Embodiment 147: The kit of embodiment 146, wherein the antibiotic is gentamicin sulfate.

Embodiment 148: The kit of any one of embodiments 140-147, wherein the medium further comprises an antifungal.

Embodiment 149: The kit of embodiment 148, wherein the antifungal is amphotericin B.

Embodiment 150: The kit of any one of embodiments 140-149, wherein the medium further comprises a salt-based solution.

Embodiment 151: The kit of embodiment 150, wherein the salt-based solution comprises Hank's Balanced Salt Solution (HBSS).

Embodiment 152: The kit of embodiment 150 or 151, wherein the salt-based solution further comprises calcium and magnesium ions.

Embodiment 153: The kit of any one of embodiments 140-152, wherein the medium further comprises an eluting solution.

Embodiment 154: The kit of embodiment 153, wherein the eluting solution comprises glycine.

Embodiment 155: The kit of embodiment 153 or 154, wherein the eluting solution further comprises at least one amphoteric amino acid.

Embodiment 156: The kit of any one of embodiments 153-155, wherein the eluting solution further comprises a sodium polyphosphate solution.

Embodiment 157: The kit of any one of embodiments 138-156, wherein the kit further comprises a unique barcode label for traceability.

Embodiment 158: The kit of embodiment 157, wherein the unique barcode label is attached to the device.

Embodiment 159: The kit of any one of embodiments 138-158, further comprising the detecting system of any one of embodiments 133-136.

All references and publications cited herein are hereby incorporated by reference. The following examples are provided to further illustrate some embodiments of the present disclosure, but are not intended to limit the scope of the disclosure; it will be understood by their exemplary nature that other procedures, methodologies, or techniques known to those skilled in the art can alternatively be used.

1. A device for collecting an aerosol sample from a subject, the device comprising:
(a) a conduit defining an inner space for passage of exhaled air, wherein the conduit comprises:
(i) a first opening for receiving the exhaled air from the subject;
(ii) a second opening for expelling at least a portion of the exhaled air; and
(iii) a long axis; and
(b) a filter for collecting the aerosol sample from the exhaled air,
wherein the filter is positioned between the first opening and the second opening and is substantially perpendicular to the long axis, and
wherein the filter:
(iv) is an electrostatically charged filter;
(v) exhibits a low airflow resistance; or
(vi) both (iv) and (v).

2. (canceled)

3. The device of claim 1, wherein the filter exhibits an airflow resistance of less than about 1.5 cm H₂O/L/sec at flow rates of about 14 L/sec.

4. The device of claim 1, wherein a length of the conduit between the first opening and the second opening is about 100 to about 150 millimeters.

5. The device of claim 1, wherein a diameter of the conduit is about 10 to about 20 millimeters.

6. (canceled)

7. The device of claim 1, wherein the filter comprises a non-woven synthetic fiber or a nanofiber.

8. The device of claim 1, wherein the filter comprises a material that is non-chemically reactive with the aerosol sample.

9. The device of claim 1, wherein the filter comprises a sensor configured to detect a molecule or a pathogen.

10. The device of claim 1, wherein the filter comprises a sensor configured to detect a bacterium, a virus, or a viral particle.

11. (canceled)

12. The device of claim 1, further comprising a detector to detect strength of breath of the subject, loudness of voice of the subject, or both.

13. The device of claim 12, wherein the detector is attached to an outside wall of the conduit.

14. The device of claim 12, wherein the detector is a detecting system that comprises
(a) a sensor for collecting information regarding the strength of the breath of the subject, the loudness of the voice of the subject, or both, and providing the information to a processor;
(b) the processor for processing the information provided by the sensor; and
(c) a structure configured to allow the sensor to attach to the conduit.

15. The device of claim 1, wherein the device is configured to capture speech-generated droplets provided by the subject while speaking.

16. A device for collecting an aerosol sample from a subject, comprising:
(a) a conduit defining an inner space for passage of exhaled air, wherein the conduit has a first opening for receiving the exhaled air from the subject;
(b) a filter positioned in the inner space; and
(c) a detector to detect strength of breath, loudness of voice, or both, wherein the detector is positioned in the inner space.

17. A device configured to collect speech-generated droplets from a subject, comprising:
(a) a conduit defining an inner space for passage of exhaled air comprising the speech-generated droplets, wherein the conduit has an opening for receiving the exhaled air from the subject; and
(b) a filter for collecting the speech-generated droplets from the exhaled air, wherein the filter is positioned in the inner space,
wherein the speech-generated droplets are produced from speaking by the subject.

18.-21. (canceled)

22. A method for collecting an aerosol sample from a subject, comprising
providing a device comprising a filter for collecting the aerosol sample from the subject; and
receiving exhaled air from the subject, wherein the exhaled air is provided by speaking by the subject.

23. The method of claim 22, further comprising detecting loudness of the speaking.

24. The method of claim 23, wherein the speaking comprises repeatedly speaking a phrase with an average loudness of about 59 dBB at a distance of about 30 centimeters.

25. The method of claim 22, wherein the aerosol sample comprises speech-generated droplets.

26. The method of claim 25, wherein the speech-generated droplets have a size from about 1 μm to about 150 μm.

27. The method of claim 22, further comprising detecting a molecule or pathogen from the filter.

28. The method of claim 27, wherein the molecule or pathogen is detected by a color signal, an optical signal, an electronic signal, a chemical signal, or by artificial intelligence.

29.-32. (canceled)
